(19) United States
(12) Patent Application Publication
(10) Pub. No.: US 2025/0236835 A1
(43) Pub. Date: Jul. 24, 2025

(54) OPEN BIOREACTOR POWER SOURCE

(71) Applicant: Amaya Etcheverry, Bonita Springs, FL (US)

(72) Inventor: Amaya Etcheverry, Bonita Springs, FL (US)

(21) Appl. No.: 18/418,754

(22) Filed: Jan. 22, 2024

(51) Int. Cl. C12M 1/00; C12M 1/12; C12M 1/34; C12M 3/00

(52) U.S. Cl. CPC C12M 43/08; C12M 21/02; C12M 23/06; C12M 23/44; C12M 29/20; C12M 41/36

(57) ABSTRACT

Presented herein are open-air bioreactor power sources. A series of bioreactor modules each containing an aqueous consortium of algal or other species are interconnected to form a power source. In certain embodiments, the bioreactor modules are configured to interface with a ground support pole. The ground support pole may contain additional support equipment such as an air pump and electrical energy storage and connection equipment.

### INTRODUCTION

This disclosure provides an open-air bioreactor configured for usage as a power source and air purifier. In certain embodiments the power source is used to supply power to a light source. In other aspects, byproducts of the bioreactor may be harvested for use as a nutrient source.

The transportation sector contributes a significant portion of air pollution. Fossil fuel internal combustion engines such as those in cars, trucks, and motorcycles emit gaseous pollutants. Tailpipe emissions can include carbon dioxide, methane, sulfurous oxides, nitrous oxides, and various other volatile organic chemicals. Other sources of hydrocarbon emissions can include crankcase blow-by and emission from leaky valves, piston rings, and gaskets. Also, handling of fossil fuels, such as spills, evaporation, poor or incomplete combustion, further contributes to the emissions load. Additional gaseous emissions can include leaks of refrigerants, such as hydrofluorocarbons, from vehicle heating, ventilation, and air conditioning (HVAC) systems.

Additionally, internal combustion engines, most notably those that run on diesel, produce and emit high levels of particulate matter. Soot particles, for example, are large clusters of carbon atoms generated in combustion chambers with aggregate sizes ranging from a few nanometers to hundreds of microns. Soot can also be formed from the lubrication of oil next to cold surfaces and from the rapid expansion cooling during a power stroke. Further particulate matter may be generated from surface friction such as from tire wear and brake pads.

According to the CDC, approximately eleven million people live within 150 meters of major roadways. Due to the proximity of their residences to a transportation corridor, occupants experience higher concentrations of emitted pollutants coupled with increased exposure times. The result is an increased risk for the development of a variety of illnesses ranging from respiratory illnesses (e.g., asthma) to cardiovascular diseases.

For a variety of reasons, transportation corridors themselves, or nearby communities, may be restricted in access to more popular renewable energy sources. Solar panels may not be widely available or affordable. The area may have a low wind speed making it unsuitable for wind turbines. A particular corridor or community may not have suitable infrastructure support for power delivery. Some areas may be particularly sensitive environments that do not lend themselves to large scale renewable energy source installations. Other communities may be too remote for grid access or connection.

Thus, there is a need for an inexpensive renewable energy source that is capable of also purifying the air surrounding the installation.

### SUMMARY

This disclosure provides for an open-air bioreactor. In certain embodiments, the open-air bioreactor may be composed of one or more modules. In an embodiment, a module may comprise a tube transparent to photosynthetically active radiation. A long axis of the tube defines a top end and a bottom end. Said top and bottom ends may be parallel to each other. A bottom piece is fitted to the bottom end of the tube. A top piece is fitted to the top of the tube. An air diffusion device is attached to and passes through the bottom piece. Air vents may be spaced at the top end of the tube. The module further comprises a cathode wire and an anode wire. In an embodiment, a consortium is contained within the tube. In an embodiment the cathode and anode are each placed within the consortium at the top and bottom ends. In certain embodiments, the module has at least one fitting configured to permit passage of the air supply through the bottom piece. In still other embodiments, the fitting is a quick-connect fitting. In still other embodiments of the module, the top piece is configured to collect and direct precipitation to the aqueous consortium. In certain embodiments, the consortium is aqueous and is composed of at least one algal species. In certain embodiments the algal species are of the genus Chlorella and Arthrospira. In still other embodiments the algal species are Chlorella vulgaris and Arthrospira platensis (commonly called “spirulina”). In still other embodiments, the tube has one or more passive gas diffusion devices. In still other embodiments, one or more consortium growth monitoring devices form a part of the module.

Certain embodiments provide for a bioreactor power source. Such a bioreactor power source may be composed of one or more modules electrically linked together. In still other embodiments the one or more modules are attached to a ground support pole. In still other embodiments, the ground support pole is configured with support bases configured to hold the bioreactor modules and provide an air supply. In still other embodiments the ground support pole is configured with one or more air inlets fluidically connected to at least one air pump. The air pump is configured to draw in air through the inlets and supply the air to one or more bioreactor modules. In still other embodiments, the bioreactor power source is configured to connect with one or more additional bioreactor power sources; the whole configured to supply electrical power to one or more electrical loads.

Certain embodiments provide for a method of providing electrical power and a supplemental protein source. In embodiments of the provided method one or more modules as described herein are provided. The one or more modules may be electrically connected to an electrical load. Generating biomass, a consortium is grown to harvest point within one or more of the modules. The biomass is harvested from one or more of the modules. In certain embodiments harvesting the biomass consists of swapping out one or more modules at the harvest point with one or more additional modules with a fresh consortium. In certain embodiments the method further specifies that one or more modules are attached to a ground support pole. In still another embodiment, an energy storage device is provided and configured to receive and store electrical energy from the one or more modules. In still other embodiments of the apparatus and method disclosed herein, the electrical load is a light source.

In an embodiment, there is provided an electrical supply and nutrient source. The electrical supply and nutrient source is comprised of a tubular module with top and bottom ends defining a central cavity within which resides an aqueous consortium composed of one or more edible species. The bottom end of the tubular module supports an air supply device. Cathode and anode wires are attached at opposing ends of the tube and configured to connect to one or more additional tubular modules with end-module cathode or anode wires further configured to supply power to an electrical load. Upon reaching a harvest point, the aqueous consortium is harvested.

### BRIEF DESCRIPTION OF THE DRAWINGS

FIG. 1. illustrates a schematic example core element of a bioreactor embodiment.

FIG. 2 illustrates the average best air quality score (graph) obtained after model bioreactors in a model environment were challenged to forty-five (45) minutes of exposure to a model air pollutant.

FIG. 3A illustrates schematically in side view one or more additional open air bioreactor modules wired together; FIG. 3B illustrates schematically in top view one or more open air bioreactor modules wired together; and FIG. 3C illustrates schematically in bottom view one or more additional open air bioreactor modules wired together.

FIG. 4 is a graphic illustrating the stable voltage output of a nine-module experimental apparatus over eighteen days.

FIG. 5 is a graph illustrating a power transfer curve for the same nine-module experimental apparatus.

FIG. 6 is a graph of the percent change in carbon dioxide level for air passed through either an aerated or a static consortium.

FIG. 7 illustrates an embodiment wherein a six-module bioreactor power system is mounted to a ground support.

### DETAILED DESCRIPTION

### Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

The term “consortium” as used herein generally refers to a microbial consortium or a microbial community. Typically, a consortium is composed of two or more bacterial or microbial organisms living symbiotically. Consortiums may be endosymbiotic, ectosymbiotic, or both. In certain instances, an algal species may be in consortium with one or more different algal, fungal, microbial, or plant species.

The transitional phrases “comprising” and “having” are open-ended and may include other, not explicitly recited, elements. An element or a plurality of elements having a particular property may include additional elements not having that property.

Embodiments disclosed with an open-ended transitional phrase such as “comprising” include, as alternative embodiments, embodiments recited with the same elements but with an intermediate or closed transitional phrase such as “consisting essentially of” or “consisting of.” As used herein, ranges and amounts can be expressed as “about” a particular value or range. About also includes the exact amount. Hence “about 5 μL” means “about 5 μL” and “5 μL.” Generally, the term “about” includes an amount that would be expected to be within experimental error or within the error expected from manufacturing, production, or experimental tolerances.

Disclosed herein are open-air bioreactors configured to serve as power sources. In certain embodiments, the power sources are used to power a lighting system, notably, streetlights. A consortium of algae within the bioreactors is supplied with an air stream. Passage of the air stream through the bioreactor results in a decrease in air pollutants. As the biomass of algae in the bioreactor increases it may be harvested and purified to provide a protein source.

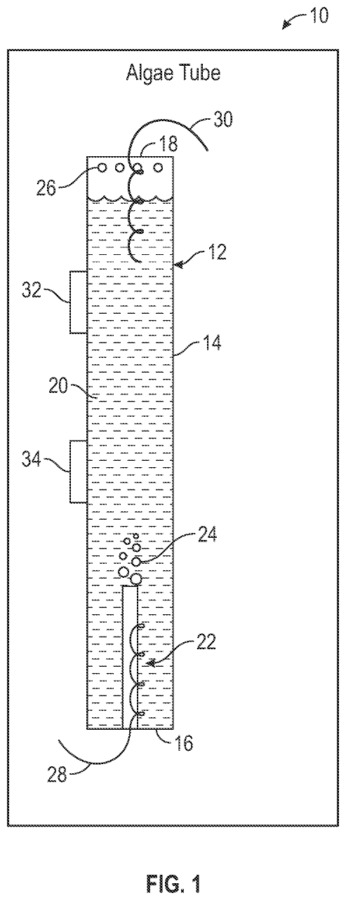

FIG. 1 illustrates a schematic example core element 10 of a bioreactor embodiment. A bioreactor housing 12 is formed by a tube 14 made of acrylic. The tube 14 is closed at a lower, bottom, end 16; and, at an upper, top, end 18, as viewed in FIG. 1. Acrylic is a suitable material due to its robust nature and high transparency to photosynthetically active radiation. One of skill in the art, however, can readily determine similar alternative materials such as quartz, polycarbonate, various polymers, and assorted glass formulations. The tube 14 forming the bioreactor wall surrounds an aqueous consortium of Arthrospira platensis and Chlorella vulgaris 20. At the bottom end 16 an air diffusion device 22 is present to provide air 24, particularly air in need of purification, into the algal consortium 20. Air introduced into the consortium passes through the length of the consortium defined by its level in the bioreactor housing. Purified air passed through the consortium 20 exits the housing through a series of air vents 26 at the top 18 of the bioreactor housing 12. Electrodes, comprising cathode and anode wires (28, 30, respectively) are installed at opposite ends of the bioreactor housing 12.

Those with skill in the art can readily imagine that air supply into the consortium 20 may be supplied by any number of air supply devices 22. By way of non-limiting examples, such means may include: porous ceramic air stones; porous metal or plastic tubes; aquarium bubblers, diffusion membranes; and, other such means as are known in the art. Although illustrated in FIG. 1 in the shape of a tube, embodiments of the apparatus may be in the shape of pucks, spirals, and other polyhedral shapes. As overall bioreactor volume or length increases one or more additional air supply means may be added. Additionally, one or more passive gas diffusion devices, such as gas permeable membranes, may be placed into the wall of the bioreactor housing 12. One or more additional agitators (not shown) may be placed into the bioreactor to assist with distribution and/or assimilation of gases into the bioreactor solution. Such agitators may take the form of propellors, spinning bars (magnetically or mechanically driven), stacked mixing blades, or air supply devices that move or otherwise agitate the bioreactor solution in an effort to enhance gas solubility.

Gas levels for chosen gases may be measured by dissolved oxygen levels in the algal consortium 20, and/or the level of carbon dioxide entering and leaving the algal consortium or bioreactor 10. Such levels may be measured through the usage of dissolved oxygen probes, infrared gas analyzers, or gas chromatographs, such as are known in the art. Gas supplies into the bioreactor may be controlled through one or more pumps (not shown) connected to the diffusion devices directly or to one or more valves, such as computer-controlled solenoid valves (not shown).

As shown in FIG. 1, the aqueous consortium 20 is one composed of Arthrospira platensis and Chlorella vulgaris algae. The algal species are inoculated into the bioreactor in a 1:1 ratio from purified stocks; although other ratios are possible such as 2:1, 1:2, 0.5:1, etc. as may be tailored to the consortium composition desired. Growth of the consortium may be monitored and tracked using one or more consortium growth monitoring devices to determine an optimal harvest time. For example, one or more optical density meters 32 and/or turbidity meters 34 may be mounted and monitored. In other embodiments, the decrease in carbon dioxide output from the system relative to the input may be calculated over time to provide an estimate of system biomass. In still other embodiments one or more colorimeters may track color change as an estimate of maturity. In still other embodiments a camera system may track changes within the system over time. Upon reaching an appropriate measure of consortium density and/or maturity, a harvest point, the consortium 20 may be harvested, and the biomass utilized as a fuel or protein source.

Although depicted in FIG. 1 with a closed top 18, embodiments may include a fully or partially open top. Additionally, the top may include one or more guides, or funnels configured to harvest and/or guide rainwater into the bioreactor and the algal consortium contained therein. Such guides may be integrated with the wall of the bioreactor or take the form of a separate add-on device configured to attach to the bioreactor.

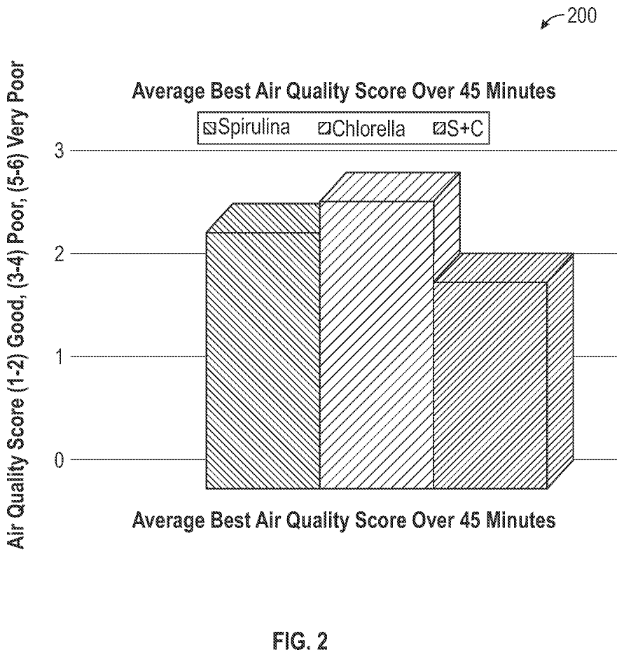

FIG. 2 illustrates the average best air quality score (graph 200) obtained after model bioreactors in a model environment were challenged to forty-five (45) minutes of exposure to a model air pollutant. A lower score is indicative of lower obtained levels of pollution and, thus, is more desirable. Surprisingly, the mixture of Arthrospira platensis and Chlorella vulgaris significantly decreased the simulated pollutant (i.e., obtained a lower score) beyond that of comparable monocultures.

As shown in FIG. 1, electrodes, comprising cathode 28 and anode 30 wires are installed at opposite ends of the bioreactor housing 12. As presented in Table 1, a variety of cathode and anode compositions may be chosen and matched to optimize voltage output. Table 1 represents voltage outputs of experimental models of embodiments of the apparatus herein. For the cathode 28, copper wire and graphite rods (carbon) were tested. For the anode 30, magnesium ribbon, magnesium rods, aluminum foil, steel, and zinc were tested. Magnesium ribbon anode with either a copper wire or graphite rod cathode returned the highest voltage. The examples provided are working examples from experimental models; those of skill in the art can recognize that cathodes 28 and anodes 30 may take any shape with larger or smaller surface areas. Each may be placed at any position in the bioreactor housing 12, including in the center, or in coaxial shapes (i.e., where a cathode and anode are coaxial to each other within the bioreactor housing 12 with the consortium 20 in fluid communication with the cathode and anode). The electrodes may be either fully or partially exposed to the algal consortium 20 (e.g., one electrode is attached to the bioreactor wall 17 while the other complementary electrode is suspended in the consortium; or, placed in an additional location on the bioreactor wall 17). Other metals such as platinum, cobalt, and molybdenum may take the place of one or more electrodes.

TABLE 1

| Cathode | Anode | Voltage (V) |
|---|---|---|
| Cu | Mg (ribbon) | 1.7 |
| Cu | Mg (rod) | 1.5 |
| Cu | Al | 0.9 |
| Cu | Steel | 0.6 |
| Cu | Zn | 0.7 |
| C | Mg (ribbon) | 1.9 |
| C | Mg (rod) | 1.8 |
| C | Al | 1.2 |
| C | Steel | 0.8 |
| C | Zn | 1.0 |
| C | Cu | 0.2 |

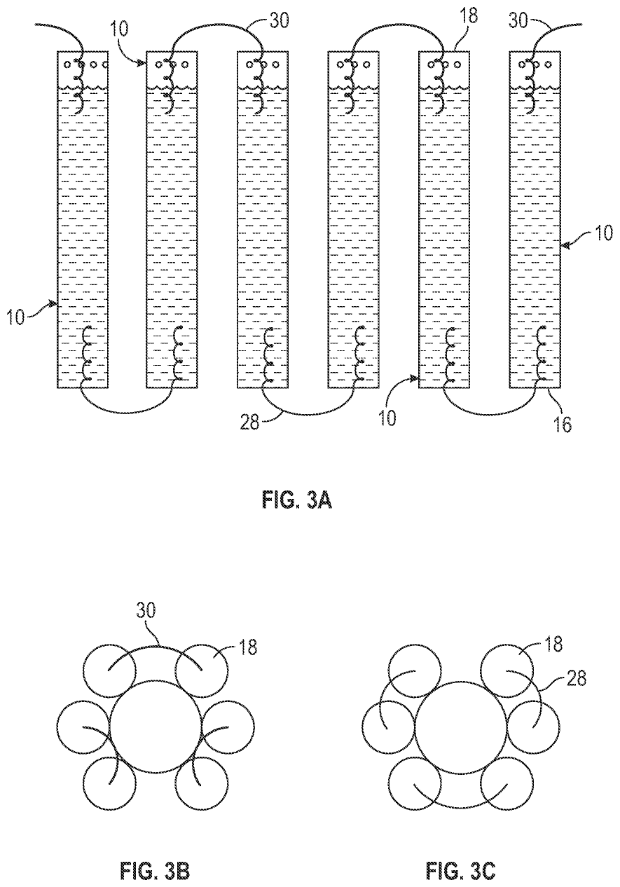

As illustrated schematically in FIG. 3 (A, side; B, top; C, bottom views), one or more additional open-air bioreactor modules 10 may be wired together, thus increasing the overall voltage available. The wiring arrangement is analogous to boosting battery voltage by wiring individual battery cells in series. As seen from the side in FIG. 3A six open-air bioreactor modules 10 are connected together with anode 30 (magnesium ribbon) at the top and cathode 28 (copper wire) at the bottom. FIGS. 3B and 3C illustrate the configuration of the connections from the top 18 (anode, 3B) and bottom 16 (cathode, 3C). One of skill in the art can readily appreciate that any number of individual bioreactor modules may be connected in such fashion. In certain embodiments, one or more fittings may be applied to top 18 and bottom 16 to permit passage of the components from the exterior of the bioreactor to the interior. Further, the end-module cathode and anode leads may then be connected to an energy storage device, such as a battery, or routed through one or more electrical components configured to, for example, provide power to an electrical load such as a light, air compressor, fans, or other electrically powered equipment.

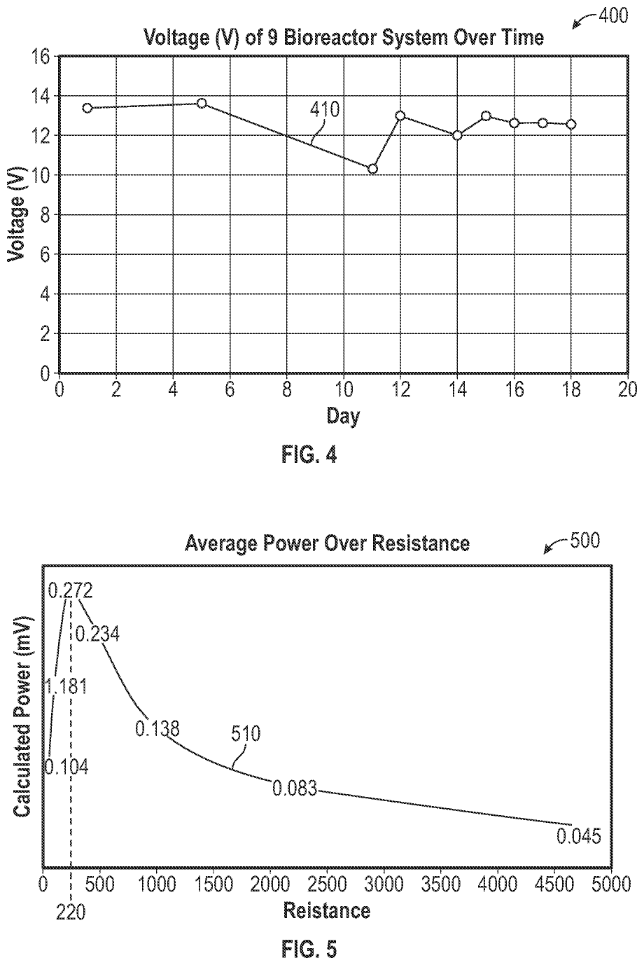

FIG. 4 is a graphic illustrating the stable voltage output 410 of a nine-module experimental apparatus over eighteen days. Voltage is stable between twelve and fourteen volts over the eighteen-day period thus confirming the longevity of the bioreactor modules and the compounded voltage. In certain embodiments, individual bioreactor power modules may be monitored for voltage output and changed or maintained upon the crossing of a lower voltage threshold.

FIG. 5 is a graph 500 illustrating a power transfer curve 510 for the same nine-module experimental apparatus. Maximum power transfer occurred when resistive load was at 220 ohms. In practice, those of skill in the art recognize that matching combinations of cathodes and anodes, consortium composition, may alter such curves with a concomitant need to match the resulting electronic load to the bioreactor power system output. Indeed, those of skill in the art may further recognize that optimizing consortium variables such as pH, electrical conductivity, dissolved oxygen, the presence of any buffers, and the like may further impact the power transfer curve.

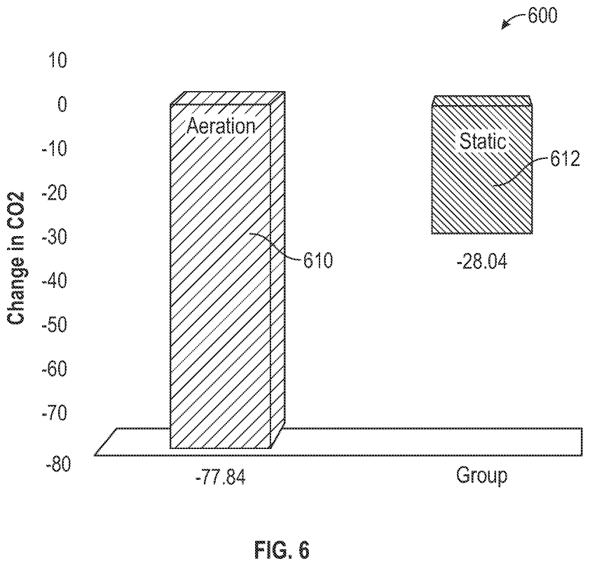

FIG. 6 is a graph 600 of the percent change in carbon dioxide level for air passed through either an aerated 610 or a static 612 consortium. Both are effective at reducing carbon dioxide, however, an aerated consortium 610 is more effective. In certain embodiments, the open-air bioreactor may remain without an active aerator while still maintaining an effective, albeit diminished, removal of carbon dioxide.

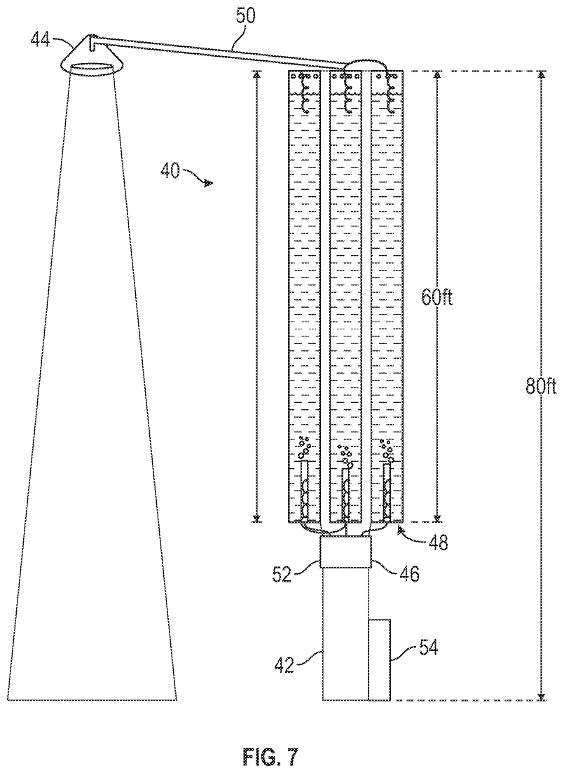

FIG. 7 illustrates an embodiment wherein a six-module bioreactor power system 40 is mounted to a ground support 42. The bioreactor power system is configured to power an elevated light source 44 and an air pump 46. As depicted, the ground support 42, in the form of a pole, has an overall length of approximately eighty feet. The bioreactor modules 10 are approximately sixty feet in length leaving the bottom most edge 48 of the system 40 still above the height of most road traffic or casual passers-by. The light 44, suspended from or integrated with a support arm 50, is then used to illuminate the surrounding area, such as a road or a courtyard. One or more additional lights may be configured at positions to illuminate one or more areas. The bioreactor power system 40 may, additionally, be configured to connect to a local power grid made up of one or more additional bioreactor power systems.

Although not depicted, one of skill in the art may readily appreciate that the air pump 46 may be housed anywhere within or on the ground support pole 42 or placed above, in the middle of, or further below the bioreactor modules. Air inlets 52 may be formed in the support pole and the support pole equipped with internal passages to convey air from the ground level to the bioreactor modules. Such air inlets may be in fluid communication with the air pump 46 which supplies air to the bioreactor power modules. In certain embodiments, air drawn in through the inlets 52 may pass through one or more filter materials. Filter materials can include, by way of non-limiting example, coconut coir, and activated charcoal.

In certain embodiments the ground support pole 42 may be further equipped with support bases configured to hold the bioreactor modules and provide an air supply. In such embodiments one or more quick-connect fittings may be fitted to a support base configured to interface with complementary fittings present on a bioreactor module. In such a configuration, the support base may additionally include one or more electrical connections allowing a properly seated bioreactor module to electrically connect with the additional one or more bioreactor modules in the overall bioreactor power system. Thus, in practice, one or more bioreactor modules may be “swapped out” for harvesting, maintenance, or other purposes.

In additional embodiments the ground support pole 42 may contain additional support equipment such as power relays, wiring, computer systems, control boards, piping, or ductwork. In further embodiments, the ground support pole may contain one or more energy storage devices 54 configured to receive and store energy generated by the bioreactor modules or from one or more additional bioreactor power systems. In still other embodiments, the ground support pole 42 may comprise an open top or a top configured to gather precipitation. Such precipitation would be directed into one or more internal or external reservoirs, said reservoirs configured with one or more feed lines configured to maintain the water level.

Finally, the written description uses examples to disclose the invention, including the best mode, and to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

Since certain changes may be made in the above-described invention, without departing from the spirit and scope of the invention herein involved, it is intended that all of the subject matter of the above description shown in the accompanying drawings shall be interpreted merely as examples illustrating the inventive concept herein and shall not be construed as limiting the invention.

1. A module, comprising:
a tube transparent to photosynthetically active radiation, a long axis of the tube defining a top end and a bottom end said top and bottom ends parallel to each other;
a bottom piece fitted to the bottom end of the tube;
a top piece fitted to the top of the tube;
an air diffusion device attached to, and passing through the bottom piece;
air vents spaced at the top end of the tube;
a cathode wire; and,
an anode wire;
wherein, a consortium is contained within the tube; and,
wherein the cathode and anode are each placed within the consortium at the top and bottom ends.

2. The module of claim 1 wherein the bottom piece further comprises at least one fitting configured to permit passage of the air supply through the bottom piece.

3. The module of claim 2 wherein the fitting is a quick-connect fitting.

4. The module of claim 1 wherein the top piece is configured to collect and direct precipitation to the aqueous consortium.

5. The module of claim 1 wherein the top piece is configured to support one or more plants.

6. The module of claim 1 wherein the consortium is aqueous and is composed of at least one algal species.

7. The module of claim 6 wherein the algal species are Arthrospira platensis and Chlorella vulgaris.

8. The module of claim 1 wherein the tube further comprises one or more passive gas diffusion devices.

9. The module of claim 1 further comprising one or more consortium growth monitoring devices.

10. A bioreactor power source, comprising:
one or more modules as claimed in claim 1 electrically linked together.

11. The bioreactor power source of claim 10 wherein the one or more modules are attached to a ground support pole.

12. The bioreactor power source of claim 11 wherein the ground support pole is configured with support bases configured to hold the bioreactor modules and provide an air supply.

13. The ground support pole of claim 11 wherein the ground support pole is configured with one or more air inlets fluidically connected to at least one air pump, said air pump further configured to draw in air through the inlets and supply the air to one or more bioreactor modules.

14. The bioreactor power source of claim 10 wherein the bioreactor power source is configured to connect with one or more additional bioreactor power sources the whole configured to supply electrical power to one or more electrical loads.

15. A method of providing electrical power and a supplemental protein source, comprising:
providing one or more modules as claimed in claim 1;
electrically connecting the one or more modules to an electrical load;
growing a consortium, generating biomass, within the one or more modules to a harvest point;
harvesting the biomass from the one or more modules.

16. The method of claim 15 wherein the harvesting further comprises swapping out one or more modules at the harvest point with one or more additional modules with a fresh consortium.

17. The method of claim 15 further comprising, attaching the one or more modules to a ground support pole.

18. The method of claim 17 further comprising, providing an energy storage device configured to receive and store electrical energy from the one or more modules.

19. The method of claim 15 wherein the electrical load is a light source.

20. An electrical power supply and protein source, comprising:
a tubular module with top and bottom ends defining a central cavity within which resides an aqueous consortium composed of one or more edible species;
the bottom end supports an air supply device;
cathode and anode wires are attached at opposing ends of the tube and configured to connect to one or more additional tubular modules with end-module cathode or anode wires further configured to supply power to an electrical load;
wherein upon reaching a harvest point the aqueous consortium is harvested.
